(19) United States
(12) Patent Application Publication
(10) Pub. No.: US 2019/0022016 A1
(43) Pub. Date: Jan. 24, 2019

(54) COMPOSITIONS FOR SUSTAINED RELEASE OF ANTI-GLAUCOMA AGENTS TO CONTROL INTRAOCULAR PRESSURE

(71) Applicant: The Johns Hopkins University, Baltimore, MD (US)

(72) Inventors: Jie Fu, Towson, MD (US); Ian Pitha, Baltimore, MD (US); Harry Quigley, Baltimore, MD (US); Justin Hanes, Baltimore, MD (US)

(21) Appl. No.: 16/081,263

(22) PCT Filed: Mar. 2, 2017
(86) PCT No.: PCT/US17/20387

§ 371 Date: Aug. 30, 2018

Related U.S. Application Data
(60) Provisional application No. 62/302,446, filed on Mar. 2, 2016.

(51) Int. Cl. A61K 9/50; A61K 9/00; A61K 31/382; A61K 31/433; A61P 27/06

(52) U.S. Cl. CPC A61K 9/5031; A61K 9/0048; A61K 31/382; A61K 31/498; A61P 27/06; A61K 2300/00; A61K 31/433

(57) ABSTRACT

Controlled release microparticular formulations for the delivery of active agents, especially for treatment of eye diseases or disorders, such as glaucoma, have been developed. These provide release of the active agent, such as a hydrophilic carbonic anhydride inhibitor, for an effective period of time such as a least one month after injection into the eye for treatment of glaucoma.

### CROSS-REFERENCE TO RELATED APPLICATIONS

This application claims benefit of and priority to U.S. Provisional Application No. 62/302,446, filed Mar. 2, 2016, which is hereby incorporated herein by reference in its entirety.

### STATEMENT OF FEDERALLY SPONSORED RESEARCH

This invention was made with government support under National Eye Institute/NIH K12-EY15025-10 and K08-EY024952. The government has certain rights in the invention.

### FIELD OF THE INVENTION

The present invention relates to polymeric controlled release formulations for the delivery of an effective amount of one or more anti-glaucoma agent, particularly those agents that lower intraocular pressure (IOP), such as dorzolamide or other carbonic anhydrase inhibitor to the eye, as well as methods of use thereof for the treatment and prevention of ocular diseases characterized by increased intraocular pressure, such as glaucoma.

### BACKGROUND OF THE INVENTION

Glaucoma is a devastating disease most often associated with elevated intraocular pressure (IOP), induced by the dysfunction of the trabecular meshwork (TM), the tissue responsible for the majority of aqueous humor outflow from the anterior chamber. Elevated IOP causes degeneration of retinal ganglion cells (RGC), resulting in visual field loss and potentially blindness.

Glaucoma affects over 70 million people worldwide and is considered a significant unmet medical need. Glaucoma is a leading cause of irreversible blindness worldwide. This number is predicted to increase to 112 million by 2040. Current therapies are focused on decreasing IOP, which reduces RGC cell degeneration and slows disease progression, even in normal-tension glaucoma. Within the next 15 years it is estimated that the glaucoma population will increase by 50% in the United States. Therefore, the identification and development of improved therapeutics and ocular delivery methods to achieve sustained IOP normalization for the treatment of glaucoma is a significant unmet need.

IOP reduction can be accomplished through topical and oral medications, laser treatment, or incisional surgery. Topically applied IOP lowering eye drops are the most commonly used, first-line glaucoma treatment. However, noncompliance with eye drop administration, especially in older patients, is a major issue in glaucoma treatment.

Eye drops lower IOP either by reducing the amount of aqueous humor produced within the eye (carbonic anhydrase inhibitors, alpha-adrenergic agonists, and beta-blockers) or by increasing fluid outflow from the eye (alpha-adrenergic agonists and prostaglandin analogues). Daily use of eye drops reduces vision loss due to glaucoma, but its success is hindered by poor patient adherence, preservative and medication toxicity, and limited bioavailability. The disincentives to ideal eye drop adherence include the fact that they provide no detectable benefit to the patient in terms of symptom relief. In addition, preservatives such as benzalkonium chloride (BAK) that are used in drop formulation can cause significant eye irritation and redness, adding additional reasons for poor drop adherence. Even when patients remember to take their eye drops, there are barriers to proper drop administration. Application of eye drops can test the manual dexterity of an aged population with glaucoma. Furthermore, once a drop is applied to the surface of the eye, there are obstacles to its effectiveness, including rapid and extensive loss by tear film dilution and drainage through the nasolacrimal duct. Given such medication clearance and the ocular barriers to drug penetration, it is not surprising that less than 3% of applied medication achieves the target intraocular tissues.

Controlled delivery of IOP lowering medications for several months after a single administration has the potential to overcome many eye drop limitations. The need for daily drop adherence is eliminated, as is the challenge of drop application. Elimination of the need for preservatives and reduction of peak drug levels could reduce ocular surface toxicity. Clinical follow-up of glaucoma patients typically occurs 2 to 4 times per year. A controlled release formulation applied by the doctor at appointments every 3 to 6 months would allow IOP control without an increase in visits.

The ideal therapeutic to reduce IOP would be an agent that specifically targets the TM, as 80-90% of aqueous humor outflow occurs through the TM and Schlemms canal. Current commercially available agents, such as timolol, a β-adrenergic receptor antagonist, and latanoprost, a prostaglandin analog, do not target the TM. Timolol functions to decrease aqueous humor production, and can have unwanted systemic respiratory and cardiac effects. Latanoprost, a prostaglandin analog, increases outflow through the uveoscleral pathway, and is responsible for only 3-35% of total aqueous humor outflow. In view of these limitations, multidrug therapy is often necessary to sufficiently lower IOP.

Therefore it is an object of the invention to provide formulations containing one or more anti-glaucoma agents, particularly those agents that lower intraocular pressure (IOP), such as carbonic anhydride inhibitors (CAI) or derivatives thereof and methods of making and using thereof that exhibit improved ocular safety and physiochemical properties.

### SUMMARY OF THE INVENTION

Formulations for the controlled delivery of one or more anti-glaucoma agents, particularly those agents that lower intraocular pressure (IOP), such as the free base form of a drug for treatment of glaucoma such as dorzolamide and brinzolamide, encapsulated in a polymeric matrix are described herein. The polymeric matrix can be formed from non-biodegradable or biodegradable polymers; however, the polymer matrix is preferably biodegradable. The polymeric matrix includes a copolymer of at least one hydrophilic polymer and a hydrophobic polymer containing COOH, COONa, or anhydride and encapsulates a therapeutic, prophylactic or diagnostic agent including a Nitrogen which complexes to the polymer. Upon administration, the agent is released over an extended period of time, either upon degradation of the polymer matrix, diffusion of the one or more inhibitors out of the polymer matrix, or a combination thereof. The solubility of the drug-polymer mixture can be controlled so as to minimize soluble drug concentration and, therefore, toxicity. The agent or agents is preferably in the free base form. The polymer-drug mixture is formed into microparticles, nanoparticles, or combinations thereof for delivery to the eye.

The one or more hydrophobic polymer segments can be any biocompatible, hydrophobic polymer or copolymer. In some cases, the hydrophobic polymer or copolymer is biodegradable. Examples of suitable hydrophobic polymers include, but are not limited to, polyesters such as polylactic acid, polyglycolic acid, or polycaprolactone, polyanhydrides, such as polysebacic anhydride, and copolymers of any of the above. In preferred embodiments, the hydrophobic polymer is a polyanhydride, such as polysebacic anhydride, poly(1,3-bis(p-carboxyphenoxy)propane, poly(1,6-bis(p-carboxyphenoxy)hexane) or a copolymer thereof.

The degradation profile of the one or more hydrophobic polymer segments may be selected to influence the release rate of the active agent in vivo. For example, the hydrophobic polymer segments can be selected to degrade over a time period from seven days to 2 years, more preferably from seven days to 56 weeks, more preferably from four weeks to 56 weeks, most preferably from eight weeks to 28 weeks.

The one or more hydrophilic polymer segments can be any hydrophilic, biocompatible, non-toxic polymer or copolymer. In certain embodiments, the one or more hydrophilic polymer segments contain a poly(alkylene glycol), such as polyethylene glycol (PEG). In particular embodiments, the one or more hydrophilic polymer segments are linear PEG chains.

In some embodiments, where both hydrophobic and hydrophilic polymer segments are present, the combined weight average molecular weight of the one or more hydrophilic polymer segments will preferably be larger than the weight average molecular weight of the hydrophobic polymer segment. In some cases, the combined weight average molecular weight of the hydrophilic polymer segments is at least five times, more preferably at least ten times, most preferably at least fifteen times, greater than the weight average molecular weight of the hydrophobic polymer segment.

The branch point, when present, can be an organic molecule which contains three or more functional groups. Preferably, the branch point will contain at least two different types of functional groups (e.g., one or more alcohols and one or more carboxylic acids, or one or more halides and one or more carboxylic acids). In such cases, the different functional groups present on the branch point can be independently addressed synthetically, permitting the covalent attachment of the hydrophobic and hydrophilic segments to the branch point in controlled stoichiometric ratios. In certain embodiments, the branch point is polycarboxylic acid, such as citric acid, tartaric acid, mucic acid, gluconic acid, or 5-hydroxybenzene-1,2,3,-tricarboxylic acid.

In certain embodiments, the polymer is formed from a single hydrophobic polymer segment and two or more hydrophilic polymer segments covalently connected via a multivalent branch point. In certain embodiments, the hydrophilic polymer segments contain a poly(alkylene glycol), such as polyethylene glycol (PEG), preferably linear PEG chains. In some embodiments, the conjugates contain between two and six hydrophilic polymer segments.

In preferred embodiments, the hydrophobic polymer is a polyanhydride, such as polysebacic anhydride or a copolymer thereof. In certain embodiments, the hydrophobic polymer segment is poly(1,6-bis(p-carboxyphenoxy)hexane-co-sebacic acid) (poly(CPH-SA) or poly(1,3-bis(p-carboxyphenoxy)propane-co-sebacic acid) (poly(CPP-SA).

The linker can be an ether (e.g., —O—), thioether (e.g., —S—), secondary amine (e.g., —NH—), tertiary amine (e.g., —NR—), secondary amide (e.g., —NHCO—; —CONH—), tertiary amide (e.g., —NRCO—; —CONR—), secondary carbamate (e.g., —OCONH—; —NHCOO—), tertiary carbamate (e.g., —OCONR—; —NRCOO—), urea (e.g., —NHCONH—; —NRCONH—; —NHCONR—, —NRCONR—), sulfinyl group (e.g., —SO—), or sulfonyl group (e.g., —SOO—), where R is, individually for each occurrence, an alkyl, cycloalkyl, heterocycloalkyl, alkylaryl, alkenyl, alkynyl, aryl, or heteroaryl group, optionally substituted with between one and five substituents individually selected from alkyl, cyclopropyl, cyclobutyl ether, amine, halogen, hydroxyl, ether, nitrile, CF3, ester, amide, urea, carbamate, thioether, carboxylic acid, and aryl, and

In certain embodiments, the branch point is a citric acid molecule, and the hydrophilic polymer segments are polyethylene glycol.

The pharmaceutical compositions can be administered to treat or prevent an ocular disease or disorder associated with increased ocular pressure. Upon administration, the agent or agents is released over an extended period of time of at least one month at concentrations which are high enough to produce therapeutic benefit, but low enough to avoid unacceptable levels of cytotoxicity.

As demonstrated by the examples, a microparticle formulation of the carbonic anhydrase inhibitor (CAI) dorzolamide that produces sustained lowering of intraocular pressure after subconjunctival injection was prepared by encapsulating the free base of the dorzolamide into poly(ethylene glycol)-poly(sebacic acid) (PEG₃-PSA) microparticles with 14.9% drug loading. In vitro drug release occurred over 12 days. Subconjunctival injection of dorzolamide (Dor) microparticles in Dutch belted rabbits reduced IOP as much as 4.06±1.53 mmHg compared to untreated fellow eyes for 35 days (P=0.02). IOP reduction after injection of Dor microparticles was significant when compared to baseline untreated IOPs (P<0.001); however, injection of blank microparticles (PEG₃-PSA) did not affect IOP (P=0.9).

Microparticle injection was associated with transient clinical vascularity and inflammatory cell infiltration in conjunctiva on histological examination. Fluorescently labeled PEG₃-PSA microparticles were detected for at least 42 days after injection, indicating that in vivo particle degradation is several-fold longer than in vitro degradation.

### BRIEF DESCRIPTION OF THE DRAWINGS

FIG. 1 is a graph of Brinzolamide and dorzolamide loading (%) as a function of TEA addition. Particle size (μm±SD) is shown on top of each column. 100 mg of PEG₃-PSA polymer was used with 20 mg of either dorzolamide or brinzolamide.

FIGS. 2A-2C are graphs of in vitro release kinetics (% over time in days) of dorzolamide and brinzolamide from PEG₃-PSA microparticles. PEG₃-PSA dorzolamide and brinzolamide microparticles using 2% PEG release drug over 12 days (2A and 2B, respectively). Release occurs over a shorter time period with 10% PEG content (2C).

FIGS. 3A-3D are graphs showing IOP reduction after subconjunctival injection of Dor microparticles. IOP reduction after topical application of 2% dorzolamide (3A) lasts several hours (n=5). Injection of microparticles without dorzolamide (PEG₃-PSA) did not reduce IOP (3B) (n=4) while subconjunctival injection of PEG₃-PSA-Dor (3C) reduced IOP for 35 days (n=7). Repeat injection of PEG₃-PSA-Dor (3D) reduced IOP (n=3). *P<0.05.

FIGS. 4A-4C are graphs of Bleb appearance and grading after microparticle injection. Bleb area (4A), bleb height 4(B), and bleb vascularity (4C) were monitored post-injection and graded using a modified version of the Moorfields Bleb Grading System (n=4).

FIG. 5 is a graph of % fluorescent signal over days post injection showing particle degradation after subconjunctival injection. Total fluorescence was followed in vivo after subconjunctival injection of PEG₃-PSA-Dox microparticles (n=4).

FIG. 6A is a graph of IOP (mmHg) over time (hours after administration) of rat eyes following topical dorzolamide eye drops (n=6). (IOP prior to topical administration was considered as 0 mmHg). (*p≤0.05)

FIG. 6B is a graph of IOP (mmHg) over time (days after administration) of rat eyes following intravitreal injection of microparticles of PEG₃-PSA loaded with dorzolamide.

FIG. 7 is a graph of IOP (mmHg) over time (days post microparticle injection) of rat eyes experiencing translimbal laser at day 2 (indicated by the arrow). (IOP of fellow, untreated, non-glaucomatous eye was considered as 0 mmHg; Y axis shows the elevation of IOP relative to the fellow eyes). Eyes injected with microparticles of PEG₃-PSA loaded with dorzolamide (n=10) are designated with squares, and eyes injected with blank microparticles (n=10) are designated with circles.

### DETAILED DESCRIPTION OF THE INVENTION

### I. Definitions

“Effective amount” or “therapeutically effective amount”, as used herein, refers to an amount of polymer effective to alleviate, delay onset of, or prevent one or more symptoms of a disease or disorder. In the case of glaucoma, the effective amount of the polymer reduces intraocular pressure (IOP).

“Biocompatible” and “biologically compatible”, as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient.

“Biodegradable Polymer” as used herein, generally refers to a polymer that will degrade or erode by enzymatic action or hydrolysis under physiologic conditions to smaller units or chemical species that are capable of being metabolized, eliminated, or excreted by the subject. The degradation time is a function of polymer composition, morphology, such as porosity, particle dimensions, and environment.

“Hydrophilic,” as used herein, refers to the property of having affinity for water. For example, hydrophilic polymers (or hydrophilic polymer segments) are polymers (or polymer segments) which are primarily soluble in aqueous solutions and/or have a tendency to absorb water. In general, the more hydrophilic a polymer is, the more that polymer tends to dissolve in, mix with, or be wetted by water.

“Hydrophobic,” as used herein, refers to the property of lacking affinity for, or even repelling water. For example, the more hydrophobic a polymer (or polymer segment), the more that polymer (or polymer segment) tends to not dissolve in, not mix with, or not be wetted by water.

Hydrophilicity and hydrophobicity can be spoken of in relative terms, such as, but not limited to, a spectrum of hydrophilicity/hydrophobicity within a group of polymers or polymer segments. In some embodiments wherein two or more polymers are being discussed, the term “hydrophobic polymer” can be defined based on the polymer's relative hydrophobicity when compared to another, more hydrophilic polymer.

“Nanoparticle”, as used herein, generally refers to a particle having a diameter, such as an average diameter, from about 10 nm up to but not including about 1 micron, preferably from 100 nm to about 1 micron. The particles can have any shape. Nanoparticles having a spherical shape are generally referred to as “nanospheres”.

“Microparticle”, as used herein, generally refers to a particle having a diameter, such as an average diameter, from about 1 micron to about 100 microns, preferably from about 1 to about 50 microns, more preferably from about 1 to about 30 microns, most preferably from about 1 micron to about 10 microns. The microparticles can have any shape. Microparticles having a spherical shape are generally referred to as “microspheres”.

“Molecular weight” as used herein, generally refers to the relative average chain length of the bulk polymer, unless otherwise specified. In practice, molecular weight can be estimated or characterized using various methods including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

“Mean particle size” as used herein, generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

“Monodisperse” and “homogeneous size distribution”, are used interchangeably herein and describe a population of nanoparticles or microparticles where all of the particles are the same or nearly the same size. As used herein, a monodisperse distribution refers to particle distributions in which 90% or more of the distribution lies within 15% of the median particle size, more preferably within 10% of the median particle size, most preferably within 5% of the median particle size.

“Pharmaceutically Acceptable”, as used herein, refers to compounds, carriers, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Branch point”, as used herein, refers to a portion of a polymer that serves to connect one or more hydrophilic polymer segments to one or more hydrophobic polymer segments.

“Implant,” as generally used herein, refers to a polymeric device or element that is structured, sized, or otherwise configured to be implanted, preferably by injection or surgical implantation, in a specific region of the body so as to provide therapeutic benefit by releasing an active agent such as a glaucoma treating agent over an extended period of time at the site of implantation. For example, intraocular implants are polymeric devices or elements that are structured, sized, or otherwise configured to be placed in the eye, preferably by injection or surgical implantation, and to treat one or more diseases or disorders of the eye by releasing the active agent over an extended period. Intraocular implants are generally biocompatible with physiological conditions of an eye and do not cause adverse side effects. Generally, intraocular implants may be placed in an eye without disrupting vision of the eye.

Ranges of values defined herein include all values within the range as well as all sub-ranges within the range. For example, if the range is defined as an integer from 0 to 10, the range encompasses all integers within the range and any and all subranges within the range, e.g., 1-10, 1-6, 2-8, 3-7, 3-9, etc.

### II. Polymer-Drug Complex

Hydrophobic drugs are delivered in a polymeric matrix formed of a copolymer of a hydrophobic polymer bound to one or more hydrophilic polymers. In some embodiments, the agent or agent is dispersed or encapsulated in the polymeric matrix for delivery to the eye. The polymeric matrix can be formed from non-biodegradable or biodegradable polymers; however, the polymer matrix is preferably biodegradable. The polymeric matrix can be formed into implants, microparticles, nanoparticles, or combinations thereof for delivery to the eye. Upon administration, the agent or agents is released over an extended period of time, either upon degradation of the polymer matrix, diffusion of the one or more inhibitors out of the polymer matrix, or a combination thereof. In certain cases, one or more hydrophilic polymer segments are attached to the one or more hydrophobic polymer segments by a branch point.

A. Polymers

The polymeric matrix includes a copolymer of at least one hydrophilic polymer and a hydrophobic polymer containing COOH, COONa, or anhydride and encapsulates a therapeutic, prophylactic or diagnostic agent including a Nitrogen which complexes to the polymer.

Hydrophobic Polymers

The hydrophobic polymer segments can be homopolymers or copolymers. In preferred embodiments, the hydrophobic polymer segment is a biodegradable polymer. In cases where the hydrophobic polymer is biodegradable, the polymer degradation profile may be selected to influence the release rate of the active agent in vivo. For example, the hydrophobic polymer segment can be selected to degrade over a time period from seven days to 2 years, more preferably from seven days to 56 weeks, more preferably from four weeks to 56 weeks, most preferably from eight weeks to 28 weeks.

Examples of suitable hydrophobic polymers include polyhydroxyacids such as poly(lactic acid), poly(glycolic acid), and poly(lactic acid-co-glycolic acids); polyhydroxyalkanoates such as poly3-hydroxybutyrate or poly4-hydroxybutyrate; polycaprolactones; poly(orthoesters); polyanhydrides, and copolymers of any of the above. In preferred embodiments, the hydrophobic polymer is a polyanhydride such as polysebacic anhydride, poly(1,3-bis(p-carboxyphenoxy)propane, poly(1,6-bis(p-carboxyphenoxy)hexane) or a copolymer thereof; poly(phosphazenes); poly(hydroxyalkanoates); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof.

In preferred embodiments, the hydrophobic polymer segment is a polyanhydride. The polyanhydride can be an aliphatic polyanhydride, an unsaturated polyanhydride, or an aromatic polyanhydride. Representative polyanhydrides include polyadipic anhydride, polyfumaric anhydride, polysebacic anhydride, polymaleic anhydride, polymalic anhydride, polyphthalic anhydride, polyisophthalic anhydride, polyaspartic anhydride, polyterephthalic anhydride, polyisophthalic anhydride, poly carboxyphenoxypropane anhydride, polycarboxyphenoxyhexane anhydride, as well as copolymers of these polyanhydrides with other polyanhydrides at different mole ratios. Other suitable polyanhydrides are disclosed in U.S. Pat. Nos. 4,757,128, 4,857,311, 4,888,176, and 4,789,724. The polyanhydride can also be a copolymer containing polyanhydride blocks.

In certain embodiments, the hydrophobic polymer segment is polysebacic anhydride. In certain embodiments, the hydrophobic polymer segment is poly(1,6-bis(p-carboxyphenoxy)hexane-co-sebacic acid) (poly(CPH-SA). In certain embodiments, the hydrophobic polymer segment is poly(1,3-bis(p-carboxyphenoxy)propane-co-sebacic acid) (poly(CPP-SA)).

The molecular weight of the hydrophobic polymer can be varied to prepare particles having properties, such as drug release rate, optimal for specific applications. The hydrophobic polymer segment can have a molecular weight of about 150 Da to 1 MDa. In certain embodiments, the hydrophobic polymer segment has a molecular weight of between about 1 kDa and about 100 kDa, more preferably between about 1 kDa and about 50 kDa, most preferably between about 1 kDa and about 25 kDa.

Hydrophilic Polymers

The one or more hydrophilic polymer segments can be any hydrophilic, biocompatible, non-toxic polymer or copolymer. Preferably, the polymer contains more than one hydrophilic polymer segment. In some embodiments, the polymer contains between two and six, more preferably between three and five, hydrophilic polymer segments. In certain embodiments, the polymer contains three hydrophilic polymer segments.

Each hydrophilic polymer segment can independently be any hydrophilic, biocompatible (i.e., it does not induce a significant inflammatory or immune response), non-toxic polymer or copolymer. Examples of suitable polymers include, but are not limited to, poly(alkylene glycols) such as polyethylene glycol (PEG), poly(propylene glycol) (PPG), and copolymers of ethylene glycol and propylene glycol, poly(oxyethylated polyol), poly(olefinic alcohol), polyvinylpyrrolidone), poly(hydroxyalkylmethacrylamide), poly(hydroxyalkylmethacrylate), poly(saccharides), poly(amino acids), poly(hydroxy acids), poly(vinyl alcohol), and copolymers, terpolymers, and mixtures thereof.

In preferred embodiments, the one or more hydrophilic polymer segments contain a poly(alkylene glycol) chain. The poly(alkylene glycol) chains may contain between 8 and 500 repeat units, more preferably between 40 and 500 repeat units. Suitable poly(alkylene glycols) include polyethylene glycol), polypropylene 1,2-glycol, poly(propylene oxide), polypropylene 1,3-glycol, and copolymers thereof. In certain embodiments, the one or more hydrophilic polymer segments are PEG chains. In such cases, the PEG chains can be linear or branched, such as those described in U.S. Pat. No. 5,932,462. In certain embodiments, the PEG chains are linear.

Each of the one or more hydrophilic polymer segments can independently have a molecular weight of about 300 Da to 1 MDa. The hydrophilic polymer segment may have a molecular weight ranging between any of the molecular weights listed above. In certain embodiments, each of the one or more hydrophilic polymer segments has a molecular weight of between about 1 kDa and about 20 kDa, more preferably between about 1 kDa and about 15 kDa, most preferably between about 1 kDa and about 10 kDa.

Branch Points

The functional groups may be any atom or group of atoms that contains at least one atom that is neither carbon nor hydrogen, with the proviso that the groups must be capable of reacting with the hydrophobic and hydrophilic polymer segments. Suitable functional groups include halogens (bromine, chlorine, and iodine); oxygen-containing functional groups such as a hydroxyls, epoxides, carbonyls, aldehydes, ester, carboxyls, and acid chlorides; nitrogen-containing functional groups such as amines and azides; and sulfur-containing groups such as thiols. The functional group may also be a hydrocarbon moiety which contains one or more non-aromatic pi-bonds, such as an alkyne, alkene, or diene. Preferably, the branch point will contain at least two different types of functional groups (e.g., one or more alcohols and one or more carboxylic acids, or one or more halides and one or more alcohols). In such cases, the different functional groups present on the branch point can be independently addressed synthetically, permitting the covalent attachment of the hydrophobic and hydrophilic segments to the branch point in controlled stoichiometric ratios.

The branch point, when present, can be an organic molecule which contains three or more functional groups. Preferably, the branch point will contain at least two different types of functional groups (e.g., one or more alcohols and one or more carboxylic acids, or one or more halides and one or more carboxylic acids or one or more amines)). In such cases, the different functional groups present on the branch point can be independently addressed synthetically, permitting the covalent attachment of the hydrophobic and hydrophilic segments to the branch point in controlled stoichiometric ratios. In certain embodiments, the branch point is polycarboxylic acid, such as citric acid, tartaric acid, mucic acid, gluconic acid, or 5-hydroxybenzene-1,2,3,-tricarboxylic acid.

Following reaction of the hydrophobic and hydrophilic polymer segments with functional groups on the branch point, the one or more hydrophobic polymer segments and the one or more hydrophilic polymer segments will be covalently joined to the branch point via linking moieties. The identity of the linking moieties will be determined by the identity of the functional group and the reactive locus of the hydrophobic and hydrophilic polymer segments (as these elements react to form the linking moiety or a precursor of the linking moiety). Examples of suitable linking moieties that connect the polymer segments to the branch point include secondary amides (—CONH—), tertiary amides (—CONR—), secondary carbamates (—OCONH—; —NHCOO—), tertiary carbamates (—OCONR—; —NRCOO—), ureas (—NHCONH—; —NRCONH—; —NHCONR—, —NRCONR—), carbinols (—CHOH—, —CROH—), ethers (—O—), and esters (—COO—, —CH₂O₂C—, CHRO₂C—), wherein R is an alkyl group, an aryl group, or a heterocyclic group. In certain embodiments, the polymer segments are connected to the branch point via an ester (—COO—, —CH₂O₂C—, CHRO₂C—), a secondary amide (—CONH—), or a tertiary amide (—CONR—), wherein R is an alkyl group, an aryl group, or a heterocyclic group.

In certain embodiments, the branch point is polycarboxylic acid, such as citric acid, tartaric acid, mucic acid, gluconic acid, or 5-hydroxybenzene-1,2,3,-tricarboxylic acid.

B. Therapeutic, Prophylactic or Diagnostic Agent

The formulations contain one or more anti-glaucoma agents. In some embodiments, the one or more agents treat glaucoma by lowering intraocular pressure (IOP). In particular embodiments, the one or more agents lower IOP by acting directly on the trabecular meshwork (TM).

Carbonic anhydrases (CA) are ubiquitous through nature and widely expressed in human tissue, including the gastrointestinal tract, kidney, liver, and skeletal muscle. Isoforms II, III, IV, and XII are present in the ciliary processes of the eye, where CA II and CA XII are involved in aqueous humor production and regulation of IOP. Becker et al. Am J Ophthalmol. 1955; 39(2 Pt 2):177-184 first showed that the systemic CA inhibitor (CAI) acetazolamide reduced IOP by 30%. Systemic CAIs are used to treat severe glaucoma; however, side effects are frequently severe and include rare but fatal aplastic anemia. Topical CAI treatment with 2% dorzolamide, available since the 1995, has no systemic side effects and reduces IOP up to 23% as monotherapy. Unfortunately, its use is limited by local eye irritation caused by the low pH and the high viscosity of its formulation. In addition, its short duration of action requires 2-3 times daily dosing, decreasing persistence and adherence. While a second topical CAI, brinzolamide, reduces IOP up to 18%, it also must be administered 2-3 times daily and blurs vision on instillation. Development of a controlled release, CAI formulation for local delivery could overcome the limitations of frequent dosing and ocular surface discomfort.

Topical CAIs are hydrophilic compounds that pose a challenge to encapsulation for controlled release. Prior attempts to formulate CAIs for controlled delivery focused on reducing side effects of eye drops or decreasing the number of times that the CAI must be applied daily. In vitro release for over 90 days and in vivo IOP lowering for at least 60 days was obtained using a polycaprolactone (PCL) blending implant to deliver dorzolamide to hypertensive rabbits. However, implant placement required surgical incisions in the conjunctiva and was associated with inflammation and fibrosis. See Natu et al., Int J Pharm. 2011, 415(1-2):73-82. doi:10.1016/j.ijpharm.2011.05.047. Optimal loading of brinzolamide and dorzolamide was obtained when the free base forms of these molecules were combined with a polyanhydride polymer.

Herein disclosed improved encapsulation of dorzolamide in a polyanhydride polymer and triethylamine (TEA) likely occurs through formation of a complex of the free base of the CAI with polyanhydride:

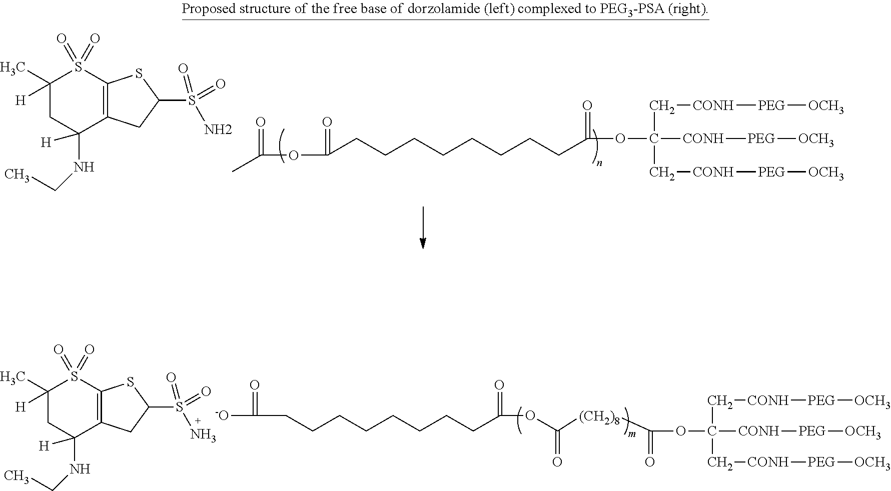

Ion pairing was used previously to improve drug loading, but not improved sufficiently by the addition of SDS or SO ion pairs, as confirmed in Table 1 in Example 2.

Representative compounds that can be complexed with polymer for delivery include brimonidine and apraclonidine, carbonic anhydrase inhibitors such as brinzolamide, acetazolamine, and dorzolamide, and other drugs containing a nitrogen, N.

Preferred weight loadings are at least 12 weight % therapeutic to total particle weight. Weight loadings are in general at least 5%, 6%, 7%, 8%, 9%, 10%, 11%, 12%, or greater, by weight.

Representative anti-glaucoma agents include prostaglandin analogs (such as travoprost, bimatoprost, and latanoprost),beta-adrenergic receptor antagonists (such as timolol, betaxolol, levobetaxolol, and carteolol), alpha-2 adrenergic receptor agonists (such as brimonidine and apraclonidine), carbonic anhydrase inhibitors (such as brinzolamide, acetazolamine, and dorzolamide), miotics (i.e., parasympathomimetics, such as pilocarpine and ecothiopate), seretonergics muscarinics, dopaminergic agonists, and adrenergic agonists (such as apraclonidine and brimonidine).

In addition to the one or more anti-glaucoma agents, particularly those agents that lower intraocular pressure (IOP), present in the polymeric particles, the formulation can contain one or more additional therapeutic, diagnostic, and/or prophylactic agents. The active agents can be a small molecule active agent or a biomolecule, such as an enzyme or protein, polypeptide, or nucleic acid. Suitable small molecule active agents include organic and organometallic compounds. In some instances, the small molecule active agent has a molecular weight of less than about 2000 g/mol, more preferably less than about 1500 g/mol, most preferably less than about 1200 g/mol. The small molecule active agent can be a hydrophilic, hydrophobic, or amphiphilic compound.

In some cases, one or more additional active agents may be encapsulated in, dispersed in, or otherwise associated with particles formed from one or more polymers. In certain embodiments, one or more additional active agents may also be dissolved or suspended in the pharmaceutically acceptable carrier.

In the case of pharmaceutical compositions for the treatment of ocular diseases, the formulation may contain one or more ophthalmic drugs. In particular embodiments, the ophthalmic drug is a drug used to treat, prevent or diagnose a disease or disorder of the posterior segment eye. Non-limiting examples of ophthalmic drugs include anti-angiogenesis agents, anti-infective agents, anti-inflammatory agents, growth factors, immunosuppressant agents, anti-allergic agents, and combinations thereof.

Representative anti-angiogenesis agents include, but are not limited to, antibodies to vascular endothelial growth factor (VEGF) such as bevacizumab (AVASTIN®) and rhuFAb V2 (ranibizumab, LUCENTIS®), and other anti-VEGF compounds including aflibercept (EYLEA®); MACUGEN® (pegaptanim sodium, anti-VEGF aptamer or EYE001) (Eyetech Pharmaceuticals); pigment epithelium derived factor(s) (PEDF); COX-2 inhibitors such as celecoxib (CELEBREX®) and rofecoxib (VIOXX®); interferon alpha; interleukin-12 (IL-12); thalidomide (THALOMID®) and derivatives thereof such as lenalidomide (REVLIMID®); squalamine; endostatin; angiostatin; ribozyme inhibitors such as ANGIOZYME® (Sirna Therapeutics); multifunctional antiangiogenic agents such as NEOVASTAT® (AE-941) (Aetema Laboratories, Quebec City, Canada); receptor tyrosine kinase (RTK) inhibitors such as sunitinib (SUTENT®); tyrosine kinase inhibitors such as sorafenib (Nexavar®) and erlotinib (Tarceva®); antibodies to the epidermal grown factor receptor such as panitumumab (VECTIBIX®) and cetuximab (ERBITUX®), as well as other anti-angiogenesis agents known in the art.

Anti-infective agents include antiviral agents, antibacterial agents, antiparasitic agents, and anti-fungal agents. Representative antiviral agents include ganciclovir and acyclovir. Representative antibiotic agents include aminoglycosides such as streptomycin, amikacin, gentamicin, and tobramycin, ansamycins such as geldanamycin and herbimycin, carbacephems, carbapenems, cephalosporins, glycopeptides such as vancomycin, teicoplanin, and telavancin, lincosamides, lipopeptides such as daptomycin, macrolides such as azithromycin, clarithromycin, dirithromycin, and erythromycin, monobactams, nitrofurans, penicillins, polypeptides such as bacitracin, colistin and polymyxin B, quinolones, sulfonamides, and tetracyclines.

In some cases, the active agent is an anti-allergic agent such as olopatadine and epinastine.

Anti-inflammatory agents include both non-steroidal and steroidal anti-inflammatory agents. Suitable steroidal active agents include glucocorticoids, progestins, mineralocorticoids, and corticosteroids.

The ophthalmic drug may be present in its neutral form, or in the form of a pharmaceutically acceptable salt. In some cases, it may be desirable to prepare a formulation containing a salt of an active agent due to one or more of the salt's advantageous physical properties, such as enhanced stability or a desirable solubility or dissolution profile.

Generally, pharmaceutically acceptable salts can be prepared by reaction of the free acid or base forms of an active agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, Md., 2000, p. 704. Examples of ophthalmic drugs sometimes administered in the form of a pharmaceutically acceptable salt include timolol maleate, brimonidine tartrate, and sodium diclofenac.

In some cases, the active agent is a diagnostic agent imaging or otherwise assessing the eye. Exemplary diagnostic agents include paramagnetic molecules, fluorescent compounds, magnetic molecules, and radionuclides, x-ray imaging agents, and contrast media.

In certain embodiments, the pharmaceutical composition contains one or more local anesthetics. Representative local anesthetics include tetracaine, lidocaine, amethocaine, proparacaine, lignocaine, and bupivacaine. In some cases, one or more additional agents, such as a hyaluronidase enzyme, is also added to the formulation to accelerate and improves dispersal of the local anesthetic.

### III. Particles and Implants for Controlled Delivery of Anti-Glaucoma Agents

Polymeric implants (e.g., rods, discs, wafers, etc.), microparticles, and nanoparticles for the controlled delivery of one or more anti-glaucoma agents, particularly those agents that lower intraocular pressure (IOP), such as ethacrynic acid (ECA) or a derivative thereof are provided, either formed of the conjugates or having the conjugates dispersed or encapsulated in a matrix. In some embodiments, the particles or implants contain the agent or agents dispersed or encapsulated in a polymeric matrix. In preferred embodiments, the particles or implants are formed from polymers containing the agent or agents which are covalently bound to a polymer.

A. Particles

Microparticles and nanoparticles can be formed from one or more species of polymers. In some cases, particles are formed from a single polymer (i.e., the particles are formed from a polymer which contains the same active agent, hydrophobic polymer segment, branch point (when present), and hydrophilic polymer segment or segments).

In other embodiments, the particles are formed from a mixture of two or more different polymers. For example, particles may be formed from two or more polymers containing the agent or agents and the same hydrophobic polymer segment, branch point (when present), and hydrophilic polymer segment or segments. In other cases, the particles are formed from two or more polymers containing the agent or agents, and different hydrophobic polymer segments, branch points (when present), and/or hydrophilic polymer segments. Such particles can be used, for example, to vary the release rate of the agent or agents.

Particles can also be formed from blends of polymers with one or more additional polymers. In these cases, the one or more additional polymers can be any of the non-biodegradable or biodegradable polymers described in Section B below, although biodegradable polymers are preferred. In these embodiments, the identity and quantity of the one or more additional polymers can be selected, for example, to influence particle stability, i.e. that time required for distribution to the site where delivery is desired, and the time desired for delivery.

Particles having an average particle size of between 10 nm and 1000 microns are useful in the compositions described herein. In preferred embodiments, the particles have an average particle size of between 10 nm and 100 microns, more preferably between about 100 nm and about 50 microns, more preferably between about 200 nm and about 50 microns. In certain embodiments, the particles are nanoparticles having a diameter of between 500 and 700 nm. The particles can have any shape but are generally spherical in shape.

In some embodiments, the population of particles formed from one or more polymers is a monodisperse population of particles. In other embodiments, the population of particles formed from one or more polymers is a polydisperse population of particles. In some instances where the population of particles formed from one or more polymers is polydisperse population of particles, greater that 50%, 60%, 65%, 70%, 75%, 80%, 85%, 90%, or 95% of the particle size distribution lies within 10% of the median particle size.

Preferably, particles formed from one or more polymers contain significant amounts of a hydrophilic polymer, such as PEG, on their surface.

Methods of Forming Microparticles and Nanoparticles

Microparticle and nanoparticles can be formed using any suitable method for the formation of polymer micro- or nanoparticles known in the art. The method employed for particle formation will depend on a variety of factors, including the characteristics of the polymers present in the polymer or polymer matrix, as well as the desired particle size and size distribution.

In circumstances where a monodisperse population of particles is desired, the particles may be formed using a method which produces a monodisperse population of nanoparticles. Alternatively, methods producing polydisperse nanoparticle distributions can be used, and the particles can be separated using methods known in the art, such as sieving, following particle formation to provide a population of particles having the desired average particle size and particle size distribution.

Common techniques for preparing microparticles and nanoparticles include, but are not limited to, solvent evaporation, hot melt particle formation, solvent removal, spray drying, phase inversion, coacervation, and low temperature casting. Suitable methods of particle formulation are briefly described below. Pharmaceutically acceptable excipients, including pH modifying agents, disintegrants, preservatives, and antioxidants, can optionally be incorporated into the particles during particle formation.

1. Solvent Evaporation

In this method, the polymer (or polymer matrix and therapeutic agent) is dissolved in a volatile organic solvent, such as methylene chloride. The organic solution containing the polymer is then suspended in an aqueous solution that contains a surface active agent such as poly(vinyl alcohol). The resulting emulsion is stirred until most of the organic solvent evaporated, leaving solid nanoparticles. The resulting nanoparticles are washed with water and dried overnight in a lyophilizer Nanoparticles with different sizes and morphologies can be obtained by this method.

Polymers which contain labile polymers, such as certain polyanhydrides, may degrade during the fabrication process due to the presence of water. For these polymers, the following two methods, which are performed in completely anhydrous organic solvents, can be used.

2. Hot Melt Particle Formation

In this method, the polymer (or polymer matrix and Therapeutic agent) is first melted, and then suspended in a non-miscible solvent (like silicon oil), and, with continuous stirring, heated to 5° C. above the melting point of the polymer. Once the emulsion is stabilized, it is cooled until the polymer particles solidify. The resulting nanoparticles are washed by decantation with a suitable solvent, such as petroleum ether, to give a free-flowing powder. The external surfaces of particles prepared with this technique are usually smooth and dense. Hot melt particle formation can be used to prepare particles containing polymers which are hydrolytically unstable, such as certain polyanhydrides. Preferably, the polymer used to prepare microparticles via this method will have an overall molecular weight of less than 75,000 Daltons.

3. Solvent Removal

Solvent removal can also be used to prepare particles from polymers that are hydrolytically unstable. In this method, the polymer (or polymer matrix and Therapeutic agent) is dispersed or dissolved in a volatile organic solvent such as methylene chloride. This mixture is then suspended by stirring in an organic oil (such as silicon oil) to form an emulsion. Solid particles form from the emulsion, which can subsequently be isolated from the supernatant. The external morphology of spheres produced with this technique is highly dependent on the identity of the polymer.

4. Spray Drying

In this method, the polymer (or polymer matrix and Therapeutic agent) is dissolved in an organic solvent such as methylene chloride. The solution is pumped through a micronizing nozzle driven by a flow of compressed gas, and the resulting aerosol is suspended in a heated cyclone of air, allowing the solvent to evaporate from the microdroplets, forming particles. Particles ranging between 0.1-10 microns can be obtained using this method.

5. Phase Inversion

Particles can be formed from polymers using a phase inversion method. In this method, the polymer (or polymer matrix and Therapeutic agent) is dissolved in a “good” solvent, and the solution is poured into a strong non solvent for the polymer to spontaneously produce, under favorable conditions, microparticles or nanoparticles. The method can be used to produce nanoparticles in a wide range of sizes, including, for example, about 100 nanometers to about 10 microns, typically possessing a narrow particle size distribution.

6. Coacervation

Techniques for particle formation using coacervation are known in the art, for example, in GB-B-929 406; GB-B-929 40 1; and U.S. Pat. Nos. 3,266,987, 4,794,000, and 4,460,563. Coacervation involves the separation of a polymer (or polymer matrix and Therapeutic agent) solution into two immiscible liquid phases. One phase is a dense coacervate phase, which contains a high concentration of the polymer, while the second phase contains a low concentration of the polymer. Within the dense coacervate phase, the polymer forms nanoscale or microscale droplets, which harden into particles. Coacervation may be induced by a temperature change, addition of a non-solvent or addition of a micro-salt (simple coacervation), or by the addition of another polymer thereby forming an interpolymer complex (complex coacervation).

7. Low Temperature Casting

Methods for very low temperature casting of controlled release microspheres are described in U.S. Pat. No. 5,019,400 to Gombotz, et al. In this method, the polymer (or polymer matrix and Therapeutic agent) is dissolved in a solvent. The mixture is then atomized into a vessel containing a liquid non-solvent at a temperature below the freezing point of the polymer solution which freezes the polymer droplets. As the droplets and non-solvent for the polymer are warmed, the solvent in the droplets thaws and is extracted into the non-solvent, hardening the microspheres.

B. Dispersions of Particles Containing One or More Anti-Glaucoma Agents in a Polymer Matrix

Particles can also be formed containing one or more anti-glaucoma agents, particularly those agents that lower IOP dispersed or encapsulated in a polymeric matrix.

Particles having an average particle size of between 10 nm and 1000 microns are useful in the compositions described herein. In preferred embodiments, the particles have an average particle size of between 10 nm and 100 microns, more preferably between about 100 nm and about 50 microns, more preferably between about 200 nm and about 50 microns. In certain embodiments, the particles are nanoparticles having a diameter of between 500 and 700 nm. The particles can have any shape but are generally spherical in shape.

C. Implants Formed from Polymers

Implants can be formed from the polymers. In preferred embodiments, the implants are intraocular implants. Suitable implants include, but are not limited to, rods, discs, wafers, and the like.

In some cases, the implants are formed from a single polymer (i.e., the implants are formed from a polymer which contains the same active agent, hydrophobic polymer segment, branch point (when present), and hydrophilic polymer segment or segments).

In other embodiments, the implants are formed from a mixture of two or more different polymers. For example, the implants are formed from two or more polymers containing one or more anti-glaucoma agents, particularly those agents that lower IOP

The implants may be of any geometry such as fibers, sheets, films, microspheres, spheres, circular discs, rods, or plaques. Implant size is determined by factors such as toleration for the implant, location of the implant, size limitations in view of the proposed method of implant insertion, ease of handling, etc.

Where sheets or films are employed, the sheets or films will be in the range of at least about 0.5 mm×0.5 mm, usually about 3 to 10 mm×5 to 10 mm with a thickness of about 0.1 to 1.0 mm for ease of handling. Where fibers are employed, the fiber diameter will generally be in the range of about 0.05 to 3 mm and the fiber length will generally be in the range of about 0.5 to 10 mm.

The size and shape of the implant can also be used to control the rate of release, period of treatment, and drug concentration at the site of implantation. Larger implants will deliver a proportionately larger dose, but depending on the surface to mass ratio, may have a slower release rate. The particular size and geometry of the implant are chosen to suit the site of implantation.

Intraocular implants may be spherical or non-spherical in shape. For spherical-shaped implants, the implant may have a largest dimension (e.g., diameter) between about 5 μm and about 2 mm, or between about 10 μm and about 1 mm for administration with a needle, greater than 1 mm, or greater than 2 mm, such as 3 mm or up to 10 mm, for administration by surgical implantation. If the implant is non-spherical, the implant may have the largest dimension or smallest dimension be from about 5 μm and about 2 mm, or between about 10 mm and about 1 mm for administration with a needle, greater than 1 mm, or greater than 2 mm, such as 3 mm or up to 10 mm, for administration by surgical implantation.

The vitreous chamber in humans is able to accommodate relatively large implants of varying geometries, having lengths of, for example, 1 to 10 mm. The implant may be a cylindrical pellet (e.g., rod) with dimensions of about 2 mm×0.75 mm diameter. The implant may be a cylindrical pellet with a length of about 7 mm to about 10 mm, and a diameter of about 0.75 mm to about 1.5 mm. In certain embodiments, the implant is in the form of an extruded filament with a diameter of about 0.5 mm, a length of about 6 mm, and a weight of approximately 1 mg. In some embodiments, the dimensions are, or are similar to, implants already approved for intraocular injection via needle: diameter of 460 microns and a length of 6 mm and diameter of 370 microns and length of 3.5 mm.

Intraocular implants may also be designed to be least somewhat flexible so as to facilitate both insertion of the implant in the eye, such as in the vitreous, and subsequent accommodation of the implant. The total weight of the implant is usually about 250 to 5000 more preferably about 500-1000 μg. In certain embodiments, the intraocular implant has a mass of about 500 μg, 750 μg, or 1000 μg.

Methods of Manufacture

Implants can be manufactured using any suitable technique known in the art. Examples of suitable techniques for the preparation of implants include solvent evaporation methods, phase separation methods, interfacial methods, molding methods, injection molding methods, extrusion methods, coextrusion methods, carver press method, die cutting methods, heat compression, and combinations thereof. Suitable methods for the manufacture of implants can be selected in view of many factors including the properties of the polymer/polymer segments present in the implant, the properties of the one or more anti-glaucoma agents, particularly those agents that lower intraocular pressure (IOP), such as ethacrynic acid (ECA) or a derivative thereof present in the implant, and the desired shape and size of the implant. Suitable methods for the preparation of implants are described, for example, in U.S. Pat. No. 4,997,652 and U.S. Patent Application Publication No. US 2010/0124565.

In certain cases, extrusion methods may be used to avoid the need for solvents during implant manufacture. When using extrusion methods, the polymer/polymer segments and the agent or agents is chosen so as to be stable at the temperatures required for manufacturing, usually at least about 85° Celsius. However, depending on the nature of the polymeric components and Therapeutic agent, extrusion methods can employ temperatures of about 25° C. to about 150° C., more preferably about 65° C. to about 130° C.

Implants may be coextruded in order to provide a coating covering all or part of the surface of the implant. Such coatings may be erodible or non-erodible, and may be impermeable, semi-permeable, or permeable to the agent or agents, water, or combinations thereof. Such coatings can be used to further control release of the agent or agents from the implant.

Compression methods may be used to make the implants. Compression methods frequently yield implants with faster release rates than extrusion methods. Compression methods may employ pressures of about 50-150 psi, more preferably about 70-80 psi, even more preferably about 76 psi, and use temperatures of about 0° C. to about 115° C., more preferably about 25° C.° C.

### IV. Pharmaceutical Formulations

Pharmaceutical formulations contain one or more species of polymers in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

Particles formed from the polymers will preferably be formulated as a solution or suspension for injection to the eye.

Pharmaceutical formulations for ocular administration are preferably in the form of a sterile aqueous solution or suspension of particles formed from one or more polymers. Acceptable solvents include, for example, water, Ringer's solution, phosphate buffered saline (PBS), and isotonic sodium chloride solution. The formulation may also be a sterile solution, suspension, or emulsion in a nontoxic, parenterally acceptable diluent or solvent such as 1,3-butanediol.

In some instances, the formulation is distributed or packaged in a liquid form. Alternatively, formulations for ocular administration can be packed as a solid, obtained, for example by lyophilization of a suitable liquid formulation. The solid can be reconstituted with an appropriate carrier or diluent prior to administration.

Solutions, suspensions, or emulsions for ocular administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers.

Solutions, suspensions, or emulsions for ocular administration may also contain one or more tonicity agents to adjust the isotonic range of the formulation. Suitable tonicity agents are well known in the art and some examples include glycerin, mannitol, sorbitol, sodium chloride, and other electrolytes.

Solutions, suspensions, or emulsions for ocular administration may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions for ocular administration may also contain one or more excipients known art, such as dispersing agents, wetting agents, and suspending agents.

### V. Methods of Use

A. Diseases and Disorders to be Treated

Controlled release dosage formulations for the delivery of one or more anti-glaucoma agents, can be used to treat or a disease or disorder associated with increased intraocular pressure. Upon administration, the agent or agents is released over an extended period of time at concentrations which are high enough to produce therapeutic benefit, but low enough to avoid cytotoxicity.

When administered to the eye, the particles release a low dose of one or more active agents over an extended period of time, preferably longer than 3, 7, 10, 15, 21, 25, 30, or 45 days. The structure of the polymer or makeup of the polymeric matrix, particle morphology, and dosage of particles administered can be tailored to administer a therapeutically effective amount of one or more active agents to the eye over an extended period of time while minimizing side effects, such as the reduction of scoptopic ERG b-wave amplitudes and/or retinal degeneration.

Typically, the particles are administered to the anterior chamber, trabecular meshwork, and Schlemms canal.

The pharmaceutical composition containing particles formed from one or more of the polymers provided herein is administered to treat or prevent an intraocular neovascular disease. In certain embodiments, the particles are formed from a polymer containing an anthracycline, such as daunorubicin or doxorubicin.

Eye diseases, particularly those characterized by ocular neovascularization, represent a significant public health concern. Intraocular neovascular diseases are characterized by unchecked vascular growth in one or more regions of the eye. Unchecked, the vascularization damages and/or obscures one or more structures in the eye, resulting in vision loss. Intraocular neovascular diseases include proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, central retinal vein occlusion (CRVO), corneal neovascularization, and retinal neovascularization (RNV). Intraocular neovascular diseases afflict millions worldwide, in many cases leading to severe vision loss and a decrease in quality of life and productivity.

Age related macular degeneration (AMD) is a leading cause of severe, irreversible vision loss among the elderly. Bressler, et al. JAMA, 291:1900-1901(2004). AMD is characterized by a broad spectrum of clinical and pathologic findings, such as pale yellow spots known as drusen, disruption of the retinal pigment epithelium (RPE), choroidal neovascularization (CNV), and disciform macular degeneration. AMD is classified as either dry (i.e., non-exudative) or wet (i.e., exudative). Dry AMD is characterized by the presence of lesions called drusen. Wet AMD is characterized by neovascularization in the center of the visual field.

Although less common, wet AMID is responsible for 80%-90% of the severe visual loss associated with AMID (Ferris, et al. Arch. Ophthamol. 102:1640-2 (1984)). The cause of AMD is unknown. However, it is clear that the risk of developing AMD increases with advancing age. AMD has also been linked to risk factors including family history, cigarette smoking, oxidative stress, diabetes, alcohol intake, and sunlight exposure.

Wet AMD is typically characterized by CNV of the macular region. The choroidal capillaries proliferate and penetrate Bruch's membrane to reach the retinal pigment epithelium (RPE). In some cases, the capillaries may extend into the subretinal space. The increased permeability of the newly formed capillaries leads to accumulation of serous fluid or blood under the RPE and/or under or within the neurosensory retina. Decreases in vision occur when the fovea becomes swollen or detached. Fibrous metaplasia and organization may ensue, resulting in an elevated subretinal mass called a disciform scar that constitutes end-stage AMD and is associated with permanent vision loss (D'Amico D J. N. Engl. J. Med. 331:95-106 (1994)).

Other diseases and disorders of the eye, such as uveitis, are also difficult to treat using existing therapies. Uveitis is a general term referring to inflammation of any component of the uveal tract, such as the iris, ciliary body, or choroid. Inflammation of the overlying retina, called retinitis, or of the optic nerve, called optic neuritis, may occur with or without accompanying uveitis.

Ocular complications of uveitis may produce profound and irreversible loss of vision, especially when unrecognized or treated improperly. The most frequent complications of uveitis include retinal detachment, neovascularization of the retina, optic nerve, or iris, and cystoid macular edema. Macular edema (ME) can occur if the swelling, leaking, and background diabetic retinopathy (BDR) occur within the macula, the central 5% of the retina most critical to vision. ME is a common cause of severe visual impairment.

There have been many attempts to treat intraocular neurovascular diseases, as well as diseases associated with chronic inflammation of the eye, with pharmaceuticals. Attempts to develop clinically useful therapies have been plagued by difficulty in administering and maintaining a therapeutically effective amount of the pharmaceutical in the ocular tissue for an extended period of time. In addition, many pharmaceuticals exhibit significant side effects and/or toxicity when administered to the ocular tissue.

Intraocular neovascular diseases are diseases or disorders of the eye that are characterized by ocular neovascularization. The neovascularization may occur in one or more regions of the eye, including the cornea, retina, choroid layer, or iris. In certain instances, the disease or disorder of the eye is characterized by the formation of new blood vessels in the choroid layer of the eye (i.e., choroidal neovascularization, CNV). In some instances, the disease or disorder of the eye is characterized by the formation of blood vessels originating from the retinal veins and extending along the inner (vitreal) surface of the retina (i.e., retinal neovascularization, RNV).

Exemplary neovascular diseases of the eye include age-related macular degeneration associated with choroidal neovascularization, proliferative diabetic retinopathy (diabetic retinopathy associated with retinal, preretinal, or iris neovascularization), proliferative vitreoretinopathy, retinopathy of prematurity, pathological myopia, von Hippel-Lindau disease, presumed ocular histoplasmosis syndrome (POHS), and conditions associated with ischemia such as branch retinal vein occlusion, central retinal vein occlusion, branch retinal artery occlusion, and central retinal artery occlusion.

The neovascularization can be caused by a tumor. The tumor may be either a benign or malignant tumor. Exemplary benign tumors include hamartomas and neurofibromas. Exemplary malignant tumors include choroidal melanoma, uveal melanoma or the iris, uveal melanoma of the ciliary body, retinoblastoma, or metastatic disease (e.g., choroidal metastasis).

The neovascularization may be associated with an ocular wound. For example, the wound may the result of a traumatic injury to the globe, such as a corneal laceration. Alternatively, the wound may be the result of ophthalmic surgery.

The polymers can be administered to prevent or reduce the risk of proliferative vitreoretinopathy following vitreoretinal surgery, prevent corneal haze following corneal surgery (such as corneal transplantation and excimer laser surgery), prevent closure of a trabeculectomy, or to prevent or substantially slow the recurrence of pterygii.

The polymers can be administered to treat or prevent an eye disease associated with inflammation. In such cases, the polymer preferably contains an anti-inflammatory agent. Exemplary inflammatory eye diseases include, but are not limited to, uveitis, endophthalmitis, and ophthalmic trauma or surgery.

The eye disease may also be an infectious eye disease, such as HIV retinopathy, toxocariasis, toxoplasmosis, and endophthalmitis.

Pharmaceutical compositions containing particles formed from one or more of the polymers can also be used to treat or prevent one or more diseases that affect other parts of the eye, such as dry eye, meibomitis, glaucoma, conjunctivitis (e.g., allergic conjunctivitis, vernal conjunctivitis, giant papillary conjunctivitis, atopic keratoconjunctivitis), neovascular glaucoma with iris neovascularization, and iritis.

B. Methods of Administration

The formulations can be administered locally to the eye by intravitreal injection (e.g., front, mid or back vitreal injection), subconjunctival injection, intracameral injection, injection into the anterior chamber via the temporal limbus, intrastromal injection, injection into the subchoroidal space, intracorneal injection, subretinal injection, and intraocular injection. In a preferred embodiment, the pharmaceutical composition is administered by intravitreal injection. Subconjunctival injection is a promising method for delivery of controlled release glaucoma medications. The subconjunctiva is a potential space that underlies the epithelial and connective tissue layers covering the sclera. Medication can be injected into this space without penetrating the structural components of the eye, thus avoiding the risks associated with intraocular injection, such as temporary blurred vision, infection, retinal detachment, and vitreous hemorrhage. Furthermore, subconjunctival delivery could favor drug penetration to the intraocular target tissues of interest, since it places the drug close to the external sclera. Transscleral rather than transcorneal drug penetration was shown to be a route of CAI delivery to the ciliary body, its site of action in lowering IOP, by Schoenwald et al., J Ocul Pharmacol Ther. 1997; 13(1):41-59. Subconjunctival delivery of ocular treatments has been utilized for decades, including triamcinolone acetonide and other steroids for inflammatory disease, see Athanasiadis, et al., J Ocul Pharmacol Ther. 2013; 29(6):516-522. doi:10.1089/jop.2012.0208, antibiotic injections for infectious disease, and anti-proliferative drugs to augment glaucoma surgery, see Van Buskirk E M., Am J Ophthalmol. 1996; 122(5):751-752. For glaucoma treatment, subconjunctival delivery of latanoprost-loaded liposomes has achieved sustained IOP reduction in normotensive rabbits, hypertensive monkeys, and in preliminary human trials (Natarajan et al. PLoS ONE. 2011; 6(9):e24513. doi:10.1371/journal.pone.0024513; Natarajan et al. ACS Nano. 2014; 8(1):419-429. doi:10.1021/nn4046024). Subconjunctival injection of controlled release formulations of brimonidine and timolol lowered IOP for 28 days and >4 months, respectively (Ng et al. Drug Deliv Transl Res. 2015; 5(5):469-479. doi:10.1007/s13346-015-0240-4; Fedorchak, et al., Exp Eye Res. 2014; 125:210-216. doi:10.1016/j.exer.2014.06.013). An important consideration when using biodegradable polymers in the subconjunctival space is the optimization of degradation rate. The ideal degradation rate would parallel drug release, ensuring that particles are not present for prolonged periods after drug has been released. This degradation profile ensures that particle build-up does not occur with repeated particle injection. The average glaucoma patient has a duration of disease in the range of 15 years. Thus, if injections were to occur 2-4 times per year, it is important that no residual amounts of injected polymer remain after each. PEG₃-PSA degradation occurs through surface erosion and in vitro drug release parallels particle degradation. In addition, in vivo degradation of fluorescently labeled PEG₃-PSA particles closely paralleled IOP lowering kinetics of Dor particles. Thus, it is hoped that there would be minimal cumulative buildup of the delivery material with multiple injections over time. Indeed, the histological study showed no detectable particle material by light microscopy 60 days after particle injection. The length of IOP lowering would be more ideally 6 months.

Implants can be administered to the eye using suitable methods for implantation known in the art. In certain embodiments, the implants are injected intravitreally using a needle, such as a 22-guage needle. Placement of the implant intravitreally may be varied in view of the implant size, implant shape, and the disease or disorder to be treated.

In some embodiments, the pharmaceutical compositions and/or implants co-administered with one or more additional active agents. “Co-administration”, as used herein, refers to administration of the controlled release formulation with one or more additional active agents within the same dosage form, as well as administration using different dosage forms simultaneously or as essentially the same time. “Essentially at the same time” as used herein generally means within ten minutes, preferably within five minutes, more preferably within two minutes, most preferably within in one minute.

Generally, the therapeutic efficacy of the compositions described herein is characterized by lowering of the IOP relative to an IOP of an eye without any treatment or to an IOP of an eye receiving vehicle or control substance (control). Typically, the lowering of the IOP relative to that of a control is lowering by 1-8 mmHg, preferably by 2-6 mmHg, and more preferably by 2-4 mmHg.

The lowering of the IOP occurs over a prolonged period of time, typically ranging from two to seven days to one to six months or more. Preferably, the reduction in IOP occurs within days and remains lower than that in the control for a period of one to six months, more preferably for a period of three to four months.

The present invention will be further understood by reference to the following non-limiting examples.

### Examples

### Example 1: Synthesis of PEG₃-PSA

Poly(ethylene glycol)-co-poly(sebacic acid) (PEG₃-PSA) was synthesized by melt polycondensation. Briefly, sebacic acid was refluxed in acetic anhydride to form sebacic acid prepolymer (Acyl-SA). Polyethylene glycol methyl ether (MW 5000, mPEG, Sigma-Aldrich, St. Louis, Mo.) was dried under vacuum to constant weight prior to use. Citric-polyethylene glycol (PEG₃) was prepared as previously described by Ben-Shabat et al. Macromol Biosci. 2006; 6(12):1019-1025. Methoxy-poly(ethylene Glycol)-amine (CH3O-PEG-NH₂) MW 5,000 (Rapp Polymer GmbH, Tubingen, Germany) (2.0 g), citric acid (Sigma-Aldrich, St. Louis, Mo.)(25.87 mg), dicyclohexylcarbodiimidde (DCC, Acros Organic, Geel, Belgium) (82.53 mg), and 4-(dimethylamino) pyridine (DMAP, Acros Organic, Geel, Belgium) (4.0 mg) were added to 10 mL methylene chloride (DCM, Fisher, Pittsburgh, Pa.), stirred overnight at room temperature, precipitated, washed with anhydrous ether (Fisher, Pittsburgh, Pa.), and dried under vacuum.

Acyl-SA and citric-PEG₃ (10% w/w) were placed into a flask under nitrogen gas and melted at 180° C. under high vacuum. Nitrogen gas was swept into the flask after 15 minutes. The reaction was allowed to proceed for 30 min. Polymers were cooled to ambient temperature, dissolved in chloroform, and precipitated into excess petroleum ether. The precipitate was collected by filtration and dried under vacuum to constant weight.

### Example 2: Preparation of Microparticles

Materials and Methods

Dorzolamide and brinzolamide microparticles were prepared by dissolving polymers (PEG₃-PSA or PLGA(1A, 2A, 4A from Lakeshare Biomaterials) with dorzolamide in dichloromethane, triethylamine (TEA) was added, and the mixture was homogenized (L4RT, Silverson Machines, East Longmeadow, Mass.) into 100 mL of an aqueous solution containing 1% polyvinyl alcohol (25 kDa, Sigma-Aldrich, St. Louis, Mo.). Particles were hardened by allowing dichloromethane to evaporate at room temperature, while stirring for 2 hours. Particles were then collected and washed three times with double distilled water via centrifugation at 6,000×g for 10 min (International Equipment Co., Needham Heights, Mass.).

Particle size distribution was determined using a Coulter Multisizer IIe (Beckman) and were resuspended in double distilled water and added dropwise to 100 ml of ISOTON II solution until the coincidence of the particles was between 8% and 10%. At least 100,000 particles were sized to determine the mean and standard deviation of particle size.

PEG₃-PSA is a polyanhydride polymer that undergoes surface erosion to deliver continuous drug release and has been previously used for ocular delivery. Particle disappearance parallels drug release due to surface erosion. Particles were suspended in phosphate buffered saline (PBS, pH 7.4) at 5 mg/mL and incubated at 37° C. on a rotating platform (140 RPM). At selected time points, supernatant was collected by centrifugation (8,000×g for 5 min) and particles were resuspended in fresh PBS. Drug content was measured by spectraphotometer.

Results

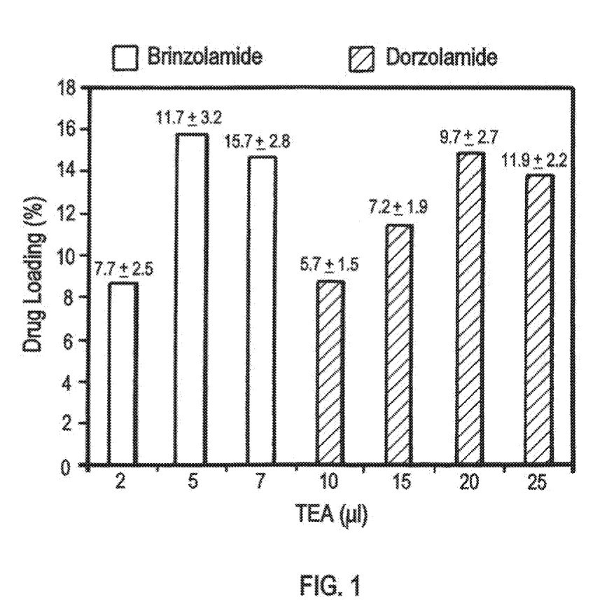

Dorzolamide and brinzolamide are hydrophilic compounds that were resistant to encapsulation into poly(lactic-co-glycolic acid)(PLGA), with loading of <1% (Table 1). Ion pairing of hydrophilic drugs with hydrophobic compounds can improve compound-polymer compatibility and drug loading, but dorzolamide ion paired with sodium dodecyl sulfate (SDS) and sodium oleate (SO) only improved drug loading to 1.5%. CAI encapsulated in PEG₃-PSA polymer was better than PLGA, and improved loading was obtained when the free base forms of dorzolamide and brinzolamide were encapsulated in PEG₃-PSA (FIG. 1). If excess TEA was added, the microparticles fragmented during synthesis. The physiochemical properties of the dorzolamide and brinzolamide microparticles are shown in Table 1. CAI encapsulation was attempted using PLGA and PEG₃-PSA polymer. Ion pairing with SDS and SO improved loading efficiency several-fold. Optimal loading was obtained with PEG₃-PSA polymer in the presence of TEA.

TABLE 1
Physiochemical properties of microparticles.

| | | | | Drug |
|---|---|---|---|---|
| | | Ion Pair | Diameter | loading |
| Drug | Formulation | (molar ratio) | (μm) | (wt. %) |
| Dorzolamide | PLGA | — | 10.9 ± 5.3 | 0.5 |
| | | SDS (0.5) | 13.3 ± 6.7 | 0.4 |
| | | SDS (1) | 15.9 ± 10.1 | 0.8 |
| | | SDS (1.5) | 13.1 ± 8.6 | 0.4 |
| | | SDS (2) | 11.5 ± 8.1 | 0.4 |
| | | SO (0.5) | 10.6 ± 4.5 | 1.3 |
| | | SO (1) | 21.6 ± 8.5 | 1.4 |
| | | SO (1.5) | 28.1 ± 10.2 | 1.5 |
| | | SO (2) | 27.9 ± 10.2 | 1.2 |
| | PEG₃-PSA | — | | 3.9 |
| | PEG₃-PSA (TEA) | — | 9.7 ± 2.7 | 14.9 |
| Brinzolamide | PLGA | — | 10.9 ± 6.2 | 1.8 |
| | PEG₃-PSA (TEA) | — | 11.7 ± 3.2 | 15.8 |

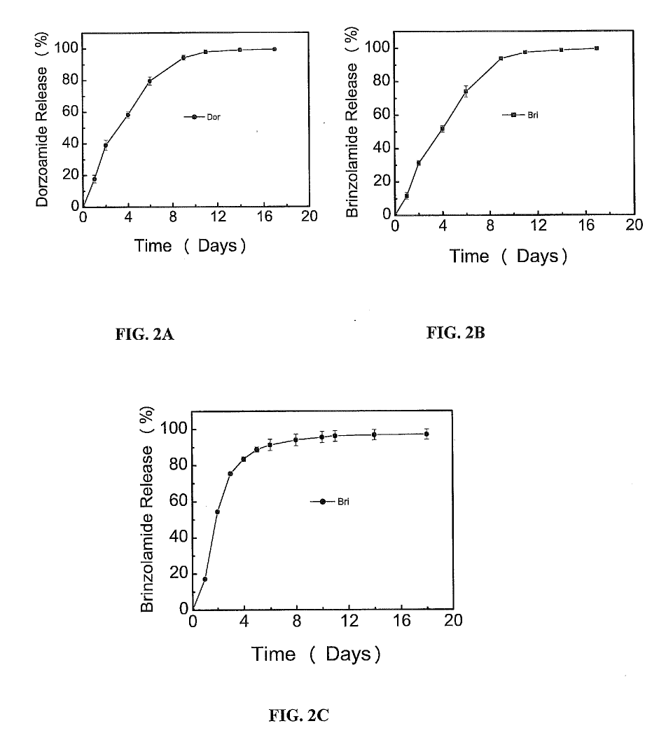

In vitro release of dorzolamide and brinzolamide from PEG₃-PSA occurred over 12 days under infinite sink conditions, with 80% released during the first 6 days (FIGS. 2A and 2B). Release for dorzolamide and brinzolamide over the initial 24 hours was 18% and 12%, respectively. Increasing the PEG content of PEG₃-PSA from 2% to 10% caused a more dramatic initial burst release and decreased the total duration of drug release (FIG. 2C). Therefore, in vivo testing was performed with Dor microparticles containing 2% PEG. 39.

### Example 3: Animal Studies in Normotensive Rabbits

Dorzolamide- and brinzolamide-loaded microparticles were designed for sustained IOP reduction after subconjunctival injection. Microparticles can be introduced into the subconjunctival space in a minimally invasive manner that may be acceptable to patients as a replacement for daily drops. To verify the efficacy and biocompatibility of the microsphere-based preparations, they were evaluated in vivo in rabbit eyes.

Materials and Methods

Dutch-belted rabbits of either sex at least 20 weeks of age were used in experimental protocols approved by the Animal Care and Use Review Board of Johns Hopkins University School of Medicine. Rabbits were handled in a manner consistent with the ARVO Statement for the Use of Animals in Ophthalmic and Vision Research, and the Guide for the Care and Use of Laboratory Animal (Institute of Laboratory Animal Resources, the Public Health Service Policy on Humane Care and Use of Laboratory Animals).

The tonometer (TonoVet; iCare, Vantaa, Finland) used for this study was calibrated for the rabbit eye. Three ex vivo rabbit eyes were cannulated by a 25-gauge needle 3 mm posterior to the limbus. The needle was connected to a manometer (DigiMano1000, Netech, Farmingdale, N.Y.) and reservoir containing balanced salt solution (BSS). The pressure set by reservoir height was verified with the manometer connected to the system and compared to the TonoVet tonometer reading. Final measurements were made after confirming stable IOP for 5 minutes. Measurements were made for manometer readings between 4 and 24 mmHg. The calibration curve for the ex vivo eyes was y=1.097×+1.74 (R²=0.98), where x=IOP reported by the TonoVet tonometer and y=manometer reading. Reported IOPs are the corrected values. For IOP measurements in this study, no anesthesia of the animal nor the eye was needed, as the instrument is well-tolerated without anesthesia.

Prior to subconjunctival injection of microparticles, anesthesia was achieved using subcutaneous injection of a mixture of ketamine (25 mg/kg) and xylazine (2.5 mg/kg). An eye drop of 1% proparacaine was followed by 5% betadine eye drop to the operative eye. Then, 0.1 ml of either Dor microparticles or blank microparticles suspended in (330 mg/me in saline with 0.25% sodium hylaluronate (HA, HA2M-5, Lifecore, Chaska, Minn.) was administered into the subconjunctival space of the superior temporal region of each eye using a 27 gauge needle. HA was added to facilitate smooth injection. Topical antibiotic ointment was administered to the eye after injection and the rabbit was examined daily for 7 days to check for signs of infection, inflammation, or irritation.

For the topical delivery group, dorzolamide eye drops (2.0% dorzolamide HCL, HiTech Pharmacal Co., Amityville, N.Y.) were administered at 9:00 am unilaterally to the upper conjunctival sac without anesthesia. Drops were administered two times separated by 5 minutes and time points reflect the time from administration of the second drop.

Before and after microparticle injection, IOP was measured with the TonoVet tonometer in awake, restrained rabbits without topical anesthesia. Each rabbit was acclimatized to the IOP measurement procedure for at least 7 days. Baseline IOP difference between right and left eyes of rabbits was averaged over three measurements taken after the acclimitization process. Anterior segment photographs of the operated eyes were performed of the area of injection, which initially appeared as an elevated zone 4 mm in diameter on the eye surface, referred to here as a bleb. A Moorfields bleb grading system designed to quantify the appearance of blebs produced by human glaucoma surgery was used to assess bleb size, height, and vascularity in all eyes (Table 2). Conjunctival morphology was graded using the Moorfields Bleb Grading System. Three masked, trained graders were used to grade photographs using this system.

TABLE 2
Description of conjunctival grading scale.

| Grade | Area | Height | Vascularity |
|---|---|---|---|
| 1 | Absent | Absent | Avascular |
| 2 | <25% upper conjunctiva | small elevation | normal |
| 3 | 25-50% upper | moderate elevation | mild |
| | conjunctiva | | |
| 4 | 50-75% upper | large elevation | moderate |
| | conjunctiva | | |
| 5 | 75-100% | | severe |

Animals were sacrificed with an intravenous overdose of Beuthanasia-D (Merck, Kenilworth, N.J.). Following enucleation, eyes were exposed to a sucrose gradient and frozen in optimal cutting temperature compound (Sakura Finetek, Torrance, Calif.) and serially cut into sections of 10 μm thickness. Sections were stained with hematoxylin-eosin (H&E).

The degradation of microparticles after subconjunctival administration was investigated by imaging fluorescently labeled particles¹ on the eye with the Xenogen IVIS spectrum optical imaging system (Caliper Life Sciences Inc., Hopkinton, Mass.). Rabbits were anesthetized as described above and PEG₃-PSA-doxorubicin (DOX) particles (33 mg in 100 μl of saline with 0.25% HA) were injected subconjunctivally into the superotemporal quadrant using a 27-gauge needle. PEG₃-PSA-DOX contain the same polymer as Dor microparticles. Additionally, they have fluorescence due to the presence of DOX. Total fluorescence at the injection site was recorded 500/600 nm and images were analyzed using Living Image 3.0 software (Caliper Lifesciences, Inc.). Retention of particles was quantified by comparing to the fluorescence counts immediately after injection to the values obtained over time.

All values are mean±standard deviation (SD). IOP reduction was calculated as the difference between the treated and untreated fellow eyes. IOP reduction following treatment was compared to mean intereye IOP difference (±SD) established on measurement of baseline, pretreatment IOPs. One-way analysis of variance test (ANOVA) was used for means. Dunnett's test (α=0.05) was performed to determine statistical significance for individual time points accounting for multiple comparisons. Area under the curve (AUC) as calculated using the trapezoid rule and statistical significance was calculated using paired t-test. P values≤0.05 were considered statistically significant.

Results

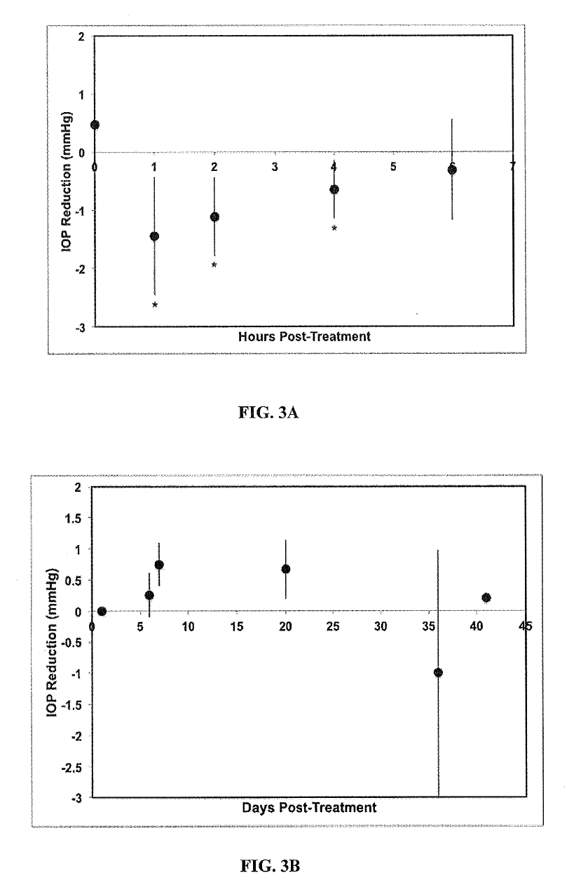

The efficacy of subconjunctivally delivered Dor microparticles was evaluated in normotensive rabbits. While IOP lowering would potentially be more dramatic in eyes that have higher than normal IOP, there is no consistent method for elevating IOP in rabbits for sustained periods of time that would leave the eye in a relatively normal physiological state. Topically delivered, 2% dorzolamide eye drops reduce IOP in normotensive rabbits only transiently. IOP was reduced after administration of 2% dorzolamide eye drops for less than 6 hours and led to a modest, but significant reduction in IOP compared to the untreated eye (one-way ANOVA, P=0.006) (FIG. 3A). A crossover effect of IOP lowering in the untreated eye by systemic absorption was not anticipated with the use of topical dorzolamide, and indeed the IOP in the untreated eye was unaffected by dorzolamide treatment. Subconjunctival injection of blank microparticles without dorzolamide (PEG₃-PSA) did not lower IOP over the course of particle degradation eye (one-way ANOVA, P=0.9) (FIG. 3B). In contrast, subconjunctival injection of Dor microparticles reduced IOP as much as 4.06±1.53 mmHg compared to untreated fellow eyes (one-way ANOVA, P<0.0001) and IOP reduction continued for 35 days after particle injection (Dunnett's test, P=0.02) (FIG. 3C). Repeat injection of PEG₃-PSA-Dor (FIG. 3D) reduced IOP.

AUC was determined for Dor and blank microparticles. There was a significant difference with Dor microparticles (−113.4±18 7 mmHg*days) and blank control microparticles (18.4±18.2 mmHg*days)(p=0.001). IOP reduction was observed on repeat injection of Dor microparticles in previously injected eyes at 60 days after initial injection and was followed through 13 days.

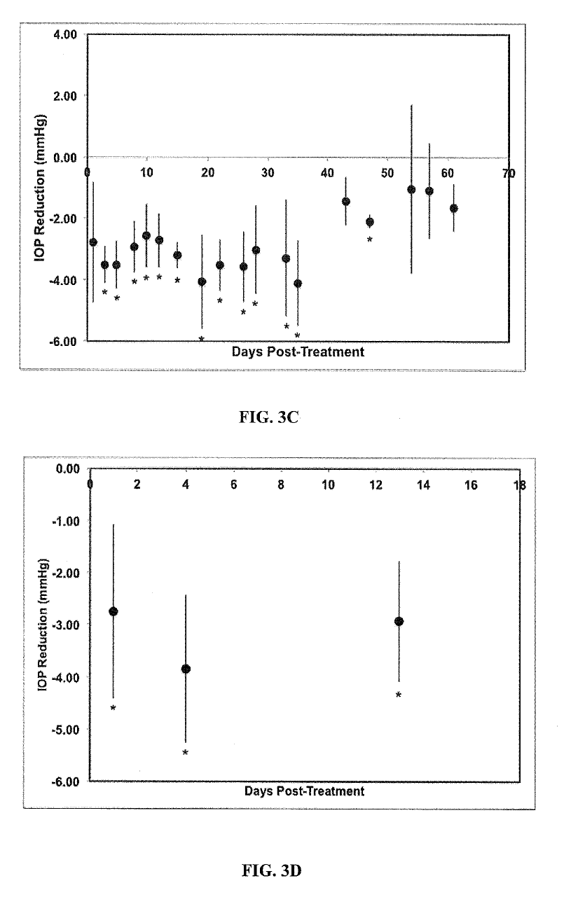

Rabbits showed no clinical signs of discomfort after subconjunctival injection of microparticles. The injected material formed an elevation (bleb) in the conjunctiva that slowly flattened over three weeks. The conjunctival vascularity over the bleb was mild, peaked at 7-14 days, and was absent 21 days after injection. FIGS. 4A-4C are graphs of Bleb appearance and grading after microparticle injection. Bleb area (4A), bleb height 4(B), and bleb vascularity (4C) were monitored post-injection and graded using a modified version of the Moorfields Bleb Grading System.

Histologic sections taken 14 days after particle injection demonstrated that the polymer was localized in the subconjunctival connective tissue with associated lymphocytes and multinucleated giant cells, demonstrating a foreign body tissue response to Dor microparticles. Some areas of one specimen had spindle shaped, basophilic fibroblasts identified.

Two weeks after injection, particles are located in the subconjunctival space with lymphocyte infiltration and polynuclear giant cells. A fibrotic response was observed in one eye with infiltration of fibroblasts. Sixty days after injection, inflammatory and fibrotic cells were no longer present.

Histologic findings consistent with inflammation and fibrosis were absent 60 days after Dor microparticle injection. There was no clinical evidence of cataract formation, aqueous humor inflammation, or abnormality in the retina, choroid and sclera. Rabbits did not demonstrate signs of ocular discomfort at any point after particle injection.

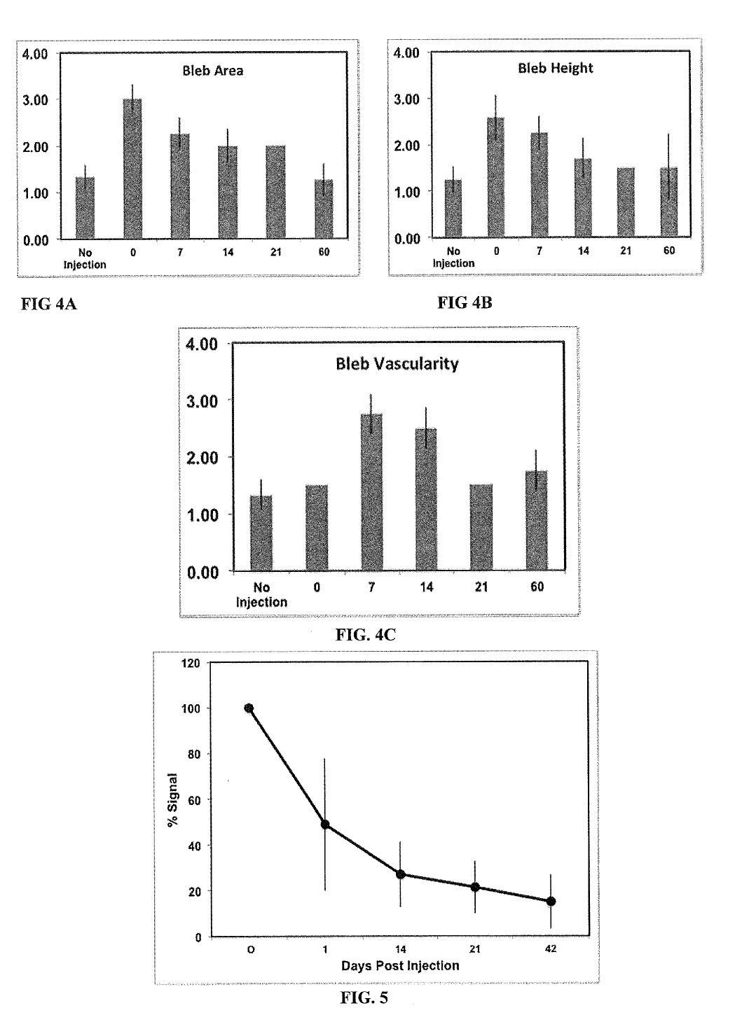

Since the duration of in vivo IOP lowering was significantly longer than of in vitro drug release, it was likely that particle degradation occurred more slowly in vivo. To corroborate this supposition, the persistence of PEG₃-PSA-Dox particles which are similar to PEG₃-PSA-Dor in size and degradation kinetics was quantified with longitudinal, in vivo whole eye imaging. After subconjunctival injection, PEG₃-PSA-Dox particle fluorescence was substantial for over one month and declined to <10% of initial fluorescence by 43 days. FIG. 5 is a graph of % fluorescent signal over days post injection showing particle degradation after subconjunctival injection. Total fluorescence was followed in vivo after subconjunctival injection of PEG₃-PSA-Dox microparticles (A) (n=4). Thus, PEG₃-PSA particle fluorescence was similar in time course to the IOP lowering effect seen with Dor microparticles. About 50% of the fluorescent signal declined over the first 24 hours after particle injection. This decline was not due to particle loss as minimal particle leakage was seen at the time of injection or on post-injection follow-up.

The results demonstrate that the biodegradable microparticle platform with high drug loading and controlled release of the CAI dorzolamide effectively lowered IOP in rabbits for over one month. The proportionate lowering observed is in a range considered clinically significant in glaucoma treatment. Dor microparticle injection was performed using a 27-gauge needle with minimal conjunctival manipulation and only mild vascularity. Improved loading of dorzolamide and brinzolamide was obtained when the drug free bases were combined with a polyanhydride (PEG₃-PSA) polymer. Microparticles can be injected into the subconjunctival space with minimal conjunctival manipulation. Additionally, the polymer components used here are classified as generally recognized as safe (GRAS) by the Food and Drug Administration and have a history of use in pharmaceutical products. Normotensive rabbits are commonly used as the experimental animal, since their eyes are similar in size to the human and they are known to respond to CAI treatment with IOP lowering.

### Example 4: Animal Studies in Normotensive Rats and in Laser-Induced Hypertensive Rats

Materials and Methods

PEG₃-PSA microparticles encapsulating dorzolamide in the presence of a base, TEA, were prepared as described in Example 2 and denoted as DPP microparticles.

Translimbal laser treatment was used to induce ocular hypertension in normotensive Wistar rats as described below and administered dorzolamide eye drops, DPP microparticles, or control microparticles lacking dorzolamide. Some eyes not treated with test agents and fellow untreated, non-glaucomatous eyes were used as control eyes in the normotensive model and the laser inducement model, respectively. In the laser inducement model, intravitreal microparticle injection was performed at day 0 and translimbal laser at day 2. IOP was monitored at least on days 1, 4, 6, 9, 11, 16, 22, and 44. On day 46, eyes were harvested for assays and quantifications of retinal ganglion cell (RGC) damage.

Results

### 1. Intravitreal Injection of DPP Microparticles Lowered IOP in Normotensive Rats.

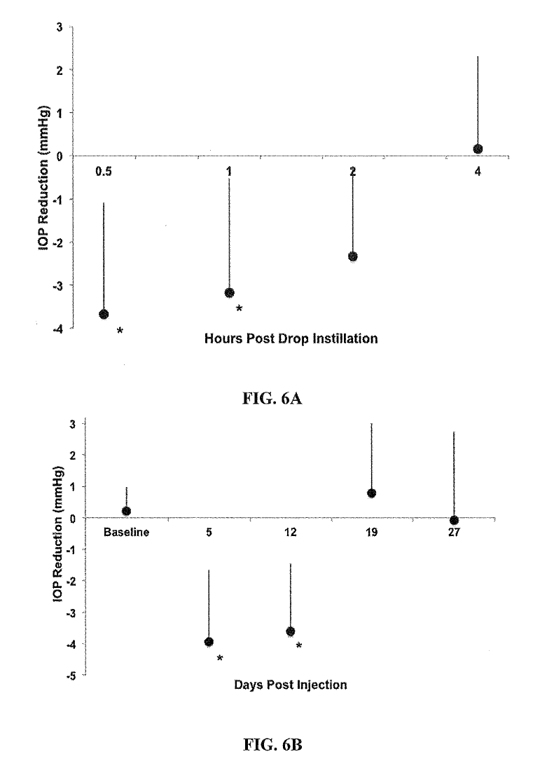

Normotensive Wistar rats had a significant but transient reduction of IOP compared to untreated eyes after delivery of dorzolamide eye drops (FIG. 6A). IOP was reduced by 3.7±2.6 mmHg at 30 minutes after the drop compared to untreated fellow eyes (p=0.01, n=6), but it was not significantly lower by 4 hours post eye-drop topical administration. In contrast, intravitreal DPP microparticle injection reduced IOP to a similar extent for a much longer duration: IOP was reduced 3.9±2.3 mm Hg (26%, p=0.01) and 3.6±2.1 mm Hg (20%, p=0.02) at 5 and 12 days after injection, respectively, in DPP microparticle injected eyes compared to control eyes (n=6)(FIG. 6B). At 19 days after microparticle injection, the difference in IOP between DPP microparticle-treated eyes and control eyes was not significant. The area under curve (AUC) of IOP reduction relative to fellow, untreated eyes was 34.1±17.0 mm Hg·days following DPP microparticle injection. In contrast the AUC after a single drop of 2% dorzolamide was 7.2913.13 mm Hg·hours. Animals did not show symptoms of eye pain. There was no hyperemia or signs of ocular inflammation on clinical exam, and particles were observed in the vitreous immediately and at 5 and 12 days after injection in all eyes except one eye. While no particle leakage was noted in this eye at the time of injection, particles were not found in this eye on clinical exam 5 days after injection. This eye was included in the analysis, though it had no IOP reduction.

### 2. DPP Microparticles Reduced Ocular Hypertensive Response to Laser Treatment.

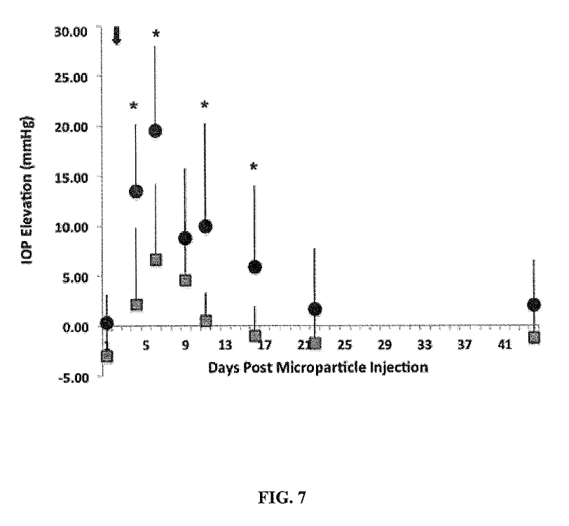

Ocular hypertension was induced by translimbal laser as described in Levkovitch-Verbin H, et al., Invest Ophthalmol Vis Sci, 43(2):402-410 (2002). All eyes received equal laser energy (0.6 W power and 0.6 second duration). DPP microparticle- and control microparticle-injected eyes received an average of 52.9±3.4 and 53.7±3.6 laser applications, respectively (p=0.61). Intravitreal DPP microparticle injection significantly reduced IOP elevation compared to untreated, fellow eyes after laser when compared to control microparticles at 4, 6, 11, and 16 days after particle injection (FIG. 7). Cumulative IOP exposure was also significantly (p=0.012) larger in eyes injected with blank microparticles (227±191 mmHg·days) compared to eyes injected with DPP microparticles (49±48 mmHg·days). Mean peak IOP relative to fellow, untreated, non-glaucomatous eyes was significantly (p=0.008) less in DPP microparticle treated eyes (22.5±6.1 mm Hg) compared to that in blank microparticle-injected eyes (34.9±6.4 mm Hg). The mean IOP elevation (relative to fellow, untreated, non-glaucomatous eyes) in the blank microparticle injection group was highest at 4 days after laser (an increase of 19.5±8.5 mm Hg), compared to an elevation of 6.7±7.5 mm Hg at the same time point in DPP injected eyes (p=0.0015).

3. DPP Microparticles Reduced Ocular Expansion after Translimbal Laser.

Experimental glaucoma in mice and rats is known to increase ocular width and length within the first week of IOP elevation. The bead-injection model of mouse glaucoma has been shown to associate with a 5-25% increase in axial length and width depending on the mouse strain tested (Cone-Kimball E, et al., Mol Vis., 19:2023-2039 (2013)). Our control injected, rat glaucoma eyes increased axial length 2.4±1.7% (p=0.04) compared with fellow un-injected eyes. This increase was not observed in DPP microparticle treated eyes: difference from fellow length was 0.3±2.2% (p=0.89) compared to fellow eyes. The group difference between control and DPP microparticle treated eyes was significant (p=0.03, t-test). There were no significant changes in axial width measurements in either glaucoma group.

### 4. DPP Microparticles Prevented RGC Loss in the Glaucoma Model.

The extent of retinal ganglion cell (RGC) damage in rat laser-induced glaucoma increases with increasing cumulative IOP exposure, higher peak IOP, and greater maximal IOP difference between a control eye and the glaucoma eye (Levkovitch-Verbin H, et al., Invest Ophthalmol Vis Sci, 43(2):402-410 (2002)). Since DPP microparticle injection significantly decreased peak IOP and cumulative IOP exposure, it was hypothesized that DPP microparticle treated eyes would be protected from loss of both RGC bodies and axons. The median axon loss in the DPP-glaucoma group was 14.1%, significantly less than the 49.6% loss in the control microparticle group (Table 3). The mean DPP group loss=24.5±31.2% (p=0.01, t test compared to fellow eyes), while the mean control microparticle group lost more than twice as many axons compared to fellow eyes (59.0±25.6%, p=0.00003). The axon loss in DPP-glaucoma group was significantly less than that in blank microparticle-glaucoma group (p=0.018).

TABLE 3
RGC axon quantifications in different treatment groups.

| | | | Control | Glaucomatous | % |
|---|---|---|---|---|---|
| Treatment | N | | (fellow) Eye | Eye | Difference |
| DPP | 9 | Mean (SD) | 117,782 | 86,170 | 24.5%* |
| particles + | | | (15,432) | (32,161) | |
| Glaucoma | | Median | 124,502 | 93,397 | 14.1%* |
| Blank | 10 | Mean (SD) | 111,073 | 45,633 | 59.0%# |
| particles + | | | (16,614) | (29,940) | |
| Glaucoma | | Median | 111,763 | 56,274 | 49.6%# |

*p = 0.01,
#p = 0.00003,
t test for difference from zero percent loss;
SD = standard deviation;
n = number of animals providing data per group.

RGC body counts from retinal whole mounts labeled with β-tubulin and DAPI demonstrated similar comparative loss to the axon counts (Table 4). The more specific label for RGCs, i.e., β-tubulin, identifies only RGC and not amacrine cells that occupy the RGC layer, the latter of which comprising about half of the neurons there. The β-tubulin data showed 61% mean loss of RGC in the control-particle group, but only 19% mean loss in the DPP-particle group. The group treated with blank microparticles suffered a significant loss according to β-tubulin quantification relative to that in fellow untreated, non-glaucomatour control eyes (p=0.012), but the loss in DPP group relative to fellow eyes was not significant (p=0.4). DAPI staining labels all nuclei in the RGC layer, both RGC and amacrines. Only RGC is believed to die in glaucoma and glaucoma models, so the potential decrease in RGC layer cells would be at most 50%. Thus, reduction in the number of DAPI-labeled nuclei would be expected to be no more than that identified by β-tubulin labeling specific to RGC. Consistent with this hypothesis, DAPI label data showed twice as many cells in the control, fellow eye RGC layer compared to β-tubulin labeling (Table 5). Likewise, the mean loss by DAPI counts in blank particle-treated glaucoma eyes was 38% compared to 6% loss in the DPP particle-treated glaucoma eyes. Again, loss in the blank particle-group was significant (p=0.021), while loss in the DPP particle-group was not (p=0.5).

TABLE 4
β-tubulin quantifications in different treatment groups.

| | | | | Glauco- | | |
|---|---|---|---|---|---|---|
| | | | Control | matous | % | P |
| Treatment | N | | Eye | Eye | Difference | value |
| DPP particles + | 5 | Mean | 770 | 625 (399) | −19% | |
| Glaucoma | | (SD) | (192) | | | |
| | | Median | 771 | 575 | −25% | 0.4 |
| Blank particles + | 5 | Mean | 839 | 324 (225) | −61% | |
| Glaucoma | | (SD) | (181) | | | |
| | | Median | 818 | 405 | −50% | 0.012 |

SD = standard deviation;
n = number of animals providing data per group

TABLE 5
DAPI quantifications in different treatment groups.

| | | | Control | Glaucomatous | % | P |
|---|---|---|---|---|---|---|
| Treatment | N | | Eye | Eye | Difference | value |
| DPP | 5 | Mean | 1,500 | 1,403 (201) | −6% | |
| particles + | | (SD) | (185) | | | |
| Glaucoma | | Median | 1440 | 1510 | 5% | 0.53 |
| Blank | 5 | Mean | 1,741 | 1,084 (244) | −38% | |
| particles + | | (SD) | (350) | | | |
| Glaucoma | | Median | 1717 | 1005 | −41% | 0.021 |

1. A polymeric matrix comprising a copolymer of at least one hydrophilic polymer and a hydrophobic polymer containing COOH, COONa, or anhydride and encapsulating a therapeutic, prophylactic or diagnostic agent including a Nitrogen which complexes to the polymer.

2. The matrix of claim 1 wherein the matrix is in the form of nanoparticles or microparticles.

3. The matrix of claim 1 wherein the polymer is a polyanhydride bound to one or more polyalkylene oxide molecules.

4. The matrix of claim 1 wherein the agent is a therapeutic for treatment of an ocular disease or disorder.

5. The matrix of claim 4 wherein the disease or disorder is glaucoma.

6. The matrix of claim 1 in the form of polymeric microparticles composed of a polyanhydride, poly(alkylene glycol), or diblock copolymer of a polyanhydride and poly(alkylene glycol).

7. The matrix of claim 1 comprising a carbonic anhydride inhibitor.

8. The matrix of claim 7 wherein the inhibitor is dorzolamide, acetazolamin, or brinzolamide.

9. The matrix of claim 1 comprising an agent selected from the group cocsisting of brimonidine, apraclonidine, and other ophthalmic drugs containing Nitrogen.

10. The matrix of claim 1 comprising at least 12 weight percent agent.

11. The matrix of claim 1 releasing agent in infinite sink conditions for at least 12 days.

12. The matrix of claim 5 providing sustained reduction of IOP after subconjunctival injection in vivo of at least 30 days

13. A method of treating a disease or disorder comprising administering a polymeric matrix comprising a copolymer of at least one hydrophilic polymer and a hydrophobic polymer containing COOH, COONa, or anhydride and encapsulating a therapeutic, prophylactic or diagnostic agent including a Nitrogen which complexes to the polymer to an individual in need thereof.

14. The method of claim 13 wherein the disease or disorder is an ocular disease or disorder.

15. The method of claim 14 wherein the disease or disorder is glaucoma.

16. The method of claim 14 wherein the matrix is microparticles administered subconjunctivally.

17. The method of claim 14 wherein the matrix is microparticles administered intravitreally.
